(19) United States
(12) Patent Application Publication
(10) Pub. No.: US 2008/0300258 A1
(43) Pub. Date: Dec. 4, 2008

(54) ANHYDROUS CIPROFLOXACIN HYDROCHLORIDE

(76) Inventors: Fabio Eduardo Silva SOUZA, Mississauga (CA); Jan Oudenes, Aurora (CA); Boris Ivanovich Gorin, Oakville (CA)

Correspondence Address: LANG MICHENER, BCE PLACE, P.O. BOX 747, SUITE 2500, 181 BAY STREET, TORONTO, ON, M5J 2T7, CA

(21) Appl. No.: 11/755,521

(22) Filed: May 30, 2007

(51) Int. Cl. A61K 31/496; C07D 295/00; A61P 31/04

(52) U.S. Cl. 514/253.06; 544/363

(57) ABSTRACT

The present invention provides a new anhydrous crystalline form of ciprofloxacin hydrochloride that is substantially free from solvent molecules, and processes of preparation thereof.

### FIELD OF INVENTION

This invention relates to ciprofloxacin hydrochloride. In particular it relates to an anhydrous crystalline form of ciprofloxacin hydrochloride and syntheses thereof.

### BRIEF DESCRIPTION OF BACKGROUND

Ciprofloxacin is chemically known as 1-cyclopropyl-6-fluoro-1,4-dihydro-4-oxo-7-(1-piperazinyl)-3-quinolinecarboxylic acid and belonging to a class of compounds called fluoroquinolones. The hydrochloride salt of ciprofloxacin, shown as structure (I) below, is a broad spectrum synthetic antibiotic effective against gram-positive organisms, such as methicillin-sensitive S. aureus, and gram-negative bacteria that include Enterobacteriaceae, Acinetobacter, Aeromonas, H. influenzae, M Catarrhalis, N. gonorrhoeae, N. meningitides, P. aeruginosa, and P. multocida. Ciprofloxacin hydrochloride also exhibits in vitro activity against other pathogens such as L. pneumophila, M. pneumoniae, and B. anthracis.

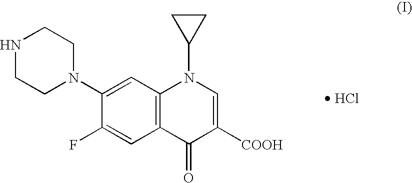

Ciprofloxacin hydrochloride exhibits its antibacterial activity by inhibiting topoisomerase II (DNA gyrase) and topoisomerase IV (both Type II topoisomerases), enzymes that are necessary for bacterial DNA replication, transcription, repair, and recombination. Topoisomerases prevent “overwinding” or excessive positive supercoiling of the DNA which can occur during and after replication. By blocking topoisomerase activity, ciprofloxacin hydrochloride inhibits DNA replication thereby resulting in cell death. Ciprofloxacin hydrochloride has been shown to be effective for the treatment of various infections including urinary tract infections, respiratory tract infections, acute sinusitis, acute uncomplicated cystitis, chronic bacterial prostates, skin and soft tissue infections, infectious diarrhea, typhoid fever, uncomplicated gonorrhea, and septicemia.

U.S. Pat. No. 4,670,444, discloses this antibiotic as the monohydrate hydrochloride salt, the form available commercially:

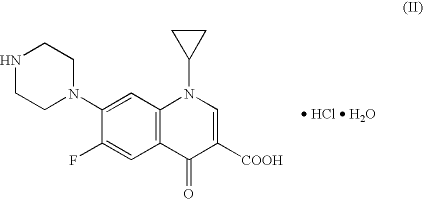

Other hydrates have since been identified and include ciprofloxacin hydrochloride 1.34-hydrate (Analytical Sciences, February 2003, Vol. 19, p. 329-330) and those reported by A. P. Kakkar et al in Drug Development in Industrial Pharmacy 1997, 23(11), 1063-1067. The same publication further discloses a dimethylformamide solvate of ciprofloxacin, along with an amorphous form.

The manufacture of pharmaceutical compositions containing ciprofloxacin hydrochloride as an active ingredient requires that the physico-chemical properties exhibited by various polymorphs and pseudopolymorphs be well understood. The varying properties of polymorphs, hydrates and solvates in turn influence the physical-chemical and mechanical properties of the resulting pharmaceutical product, and in particular, its solubility and dissolution. Dissolution studies have shown that the hydrated form of ciprofloxacin exhibits a significantly lower dissolution rate than the anhydrous form. To optimize bioavailability and the pharmacokinetic profile of the pharmaceutical formulation, the anhydrous form of ciprofloxacin hydrochloride is therefore preferred.

While solvates of ciprofloxacin hydrochloride are known (A. P. Kakkar et al. disclose, for example, a dimethylformamide solvate of ciprofloxacin hydrochloride), their use in clinical applications is less popular because of the risk of toxicity of the solvent on the delicate biological environment.

### SUMMARY OF THE INVENTION

The present invention provides a new anhydrous crystalline form of ciprofloxacin hydrochloride that is substantially free from solvent molecules, and processes of preparation thereof.

### BRIEF DESCRIPTION OF THE DRAWINGS

FIG. 1 is an IR spectrum of an anhydrous ciprofloxacin hydrochloride according to the invention;

FIG. 2 is an IR spectrum of ciprofloxacin hydrochloride monohydrate of the prior art;

FIG. 3 is a powder X-ray diffraction spectrum of both an anhydrous polymorph of the invention (bottom spectrum) and the ciprofloxacin hydrochloride monohydrate (top spectrum).

FIG. 4 is a table of the powder X-ray diffraction pattern intensities of the polymorph according to the invention.

### DETAILED DESCRIPTION OF THE PREFERRED EMBODIMENT

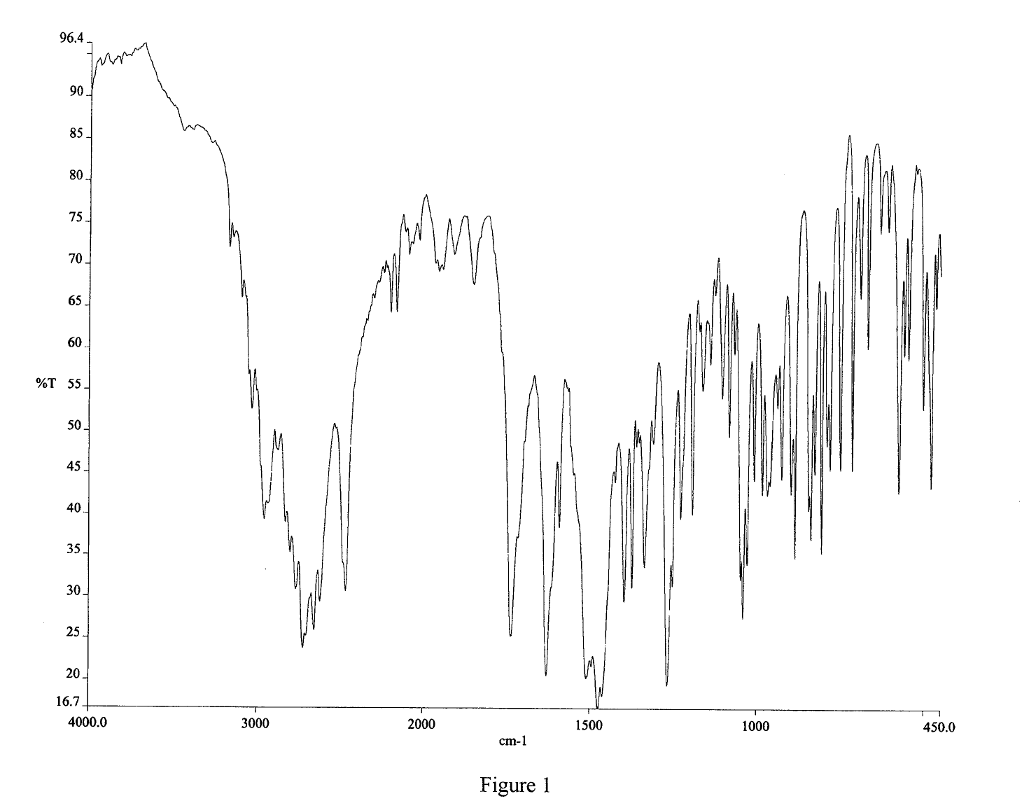

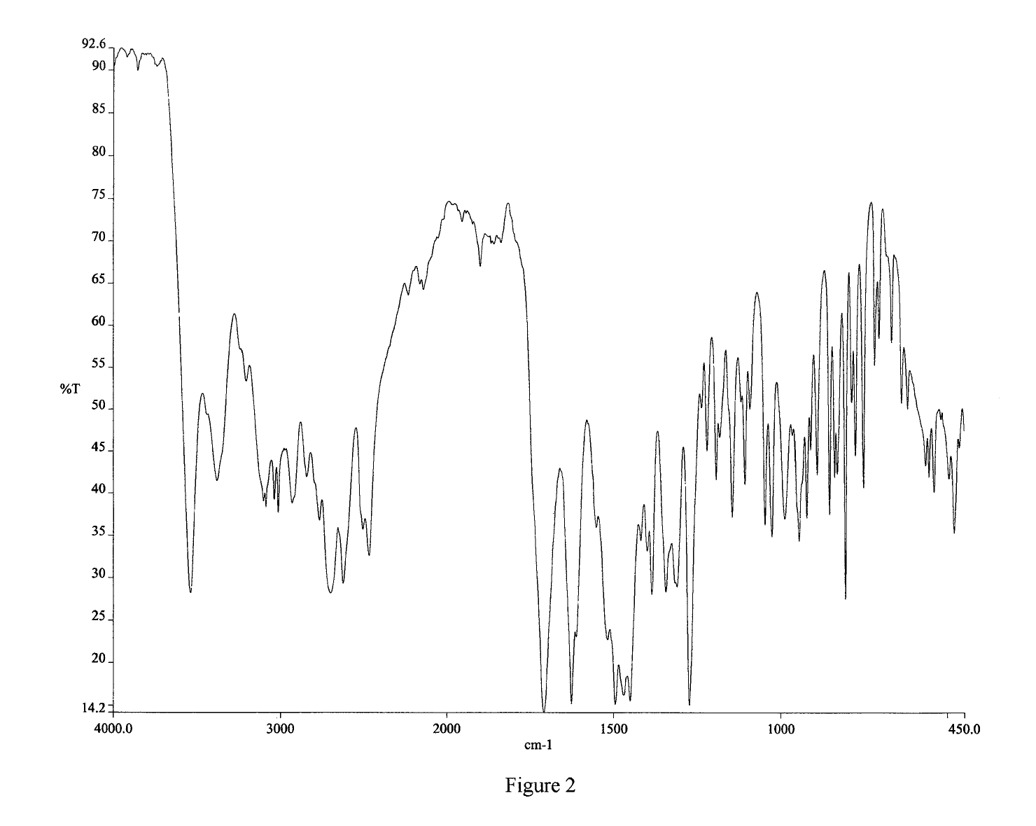

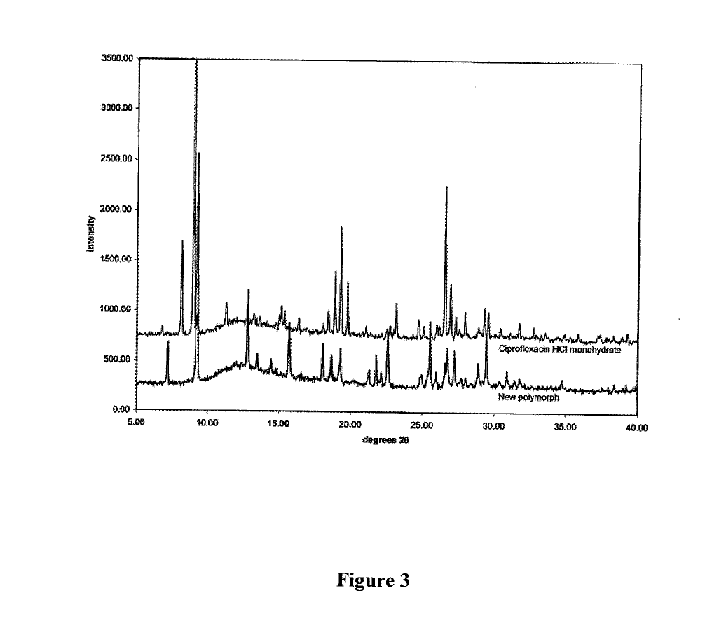

The invention provides a crystalline form of ciprofloxacin hydrochloride that is anhydrous (containing a residual water content of between 0 and about 0.4% and unsolvated (between 0 and about 2% solvent). A comparison of the infrared spectra in FIGS. 1 and 2, shows that the novel crystalline anhydrous ciprofloxacin hydrochloride is distinct from the previously known ciprofloxacin hydrochloride monohydrate. In particular, it is to be noted that the water bands are clearly visible in the spectrum of the hydrate form (FIG. 2) compared to their absence in FIG. 1. Similarly, the powder X-ray diffraction pattern of the novel anhydrous form is clearly distinct from that of ciprofloxacin hydrochloride monohydrate. as shown on accompanying FIG. 3, lower trace. Characterizing XPRD peaks for the novel polymorph of the invention are exhibited at 7.22, 9.26, 12.80, 15.71, 21.77 and 29.42 degrees 2-θ,

Moreover, the ciprofloxacin hydrochloride polymorph of the present invention shows outstanding stability characteristics. Even after eight weeks of storage at 40 degrees C., at a relative humidity of 75%, No water intake was observed, and no significant decomposition was observed—merely a single, harmless impurity at 0.06%. In addition, the polymorph of the invention has a much faster dissolution rate than the commercially accepted monohydrate, making for greater bioavailability. This combination of stability and bioavailability makes the polymorph of the invention an attractive candidate for commercialization.

According to another aspect of the invention, the anhydrous crystalline form of ciprofloxacin hydrochloride can be prepared in at least two different ways: (1) by suspending the ciprofloxacin hydrochloride monohydrate in a solvent followed by filtration of resulting precipitation; (2) by equilibrating ciprofloxacin hydrochloride monohydrate in a saturated atmosphere of a non-aqueous solvent for a period of time, for example 1-5 days.

Suitable solvents for the preparation of ciprofloxacin hydrochloride anhydrate in accordance with the invention, are biocompatible solvents. Preferably, solvents used have an affinity for water and, even more preferably, can form an azeotropic mixture with water. Examples of suitable solvents include biocompatible lower alcohols, for example ethanol, 1-butanol and 2-butanol. The residual solvent content should be below 0.5% w/w, and the water content should be below 0.25% w/w. This can be achieved by drying under vacuum at 60 degrees C. as described in the Examples below.

In the first method, ciprofloxacin hydrochloride monohydrate is suspended in solvent, and the suspension is then stirred vigorously, preferably from 2-24 hours, and may be heated then cooled to room temperature. The resulting precipitate is then collected by filtration, preferably by vacuum filtration to give anhydrous ciprofloxacin hydrochloride.

In the second method, ciprofloxacin hydrochloride monohydrate is placed in a closed chamber that is saturated with non-aqueous biocompatible solvent vapour, preferably that of a lower alcohol, for an extended period of time, preferably a few days.

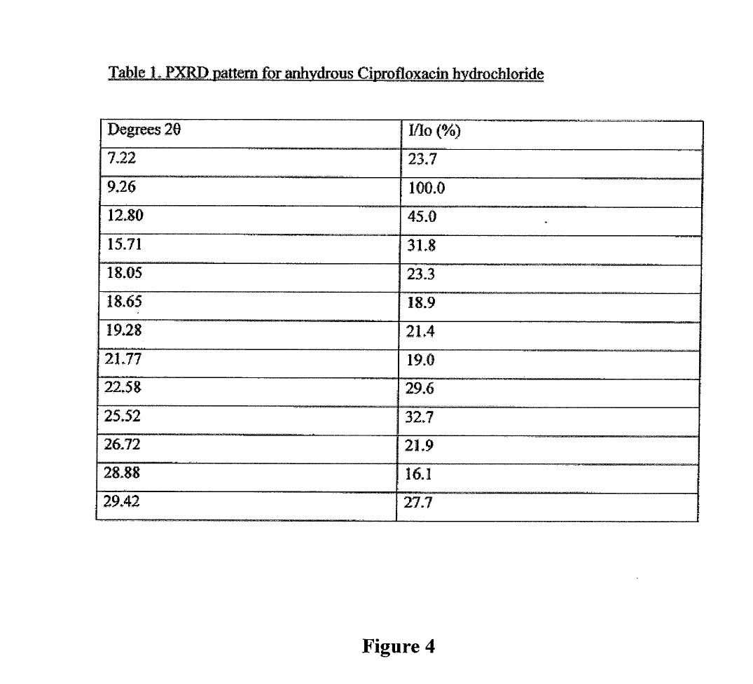

Regardless of which method is employed, the same anhydrous unsolvated form of ciprofloxacin hydrochloride is obtained that is characterized by the infrared spectrum of FIG. 1 and the powder X-ray diffraction pattern shown in FIGS. 3 and 4.

### EXAMPLES

The following are specific examples to illustrate the preparation of anhydrous ciprofloxacin hydrochloride according to the invention.

### Example 1

Ciprofloxacin hydrochloride monohydrate (50.2 g, 130 mmol) was suspended in absolute ethanol (350 mL) and vigorously stirred for 24 hours. The solids were collected by filtration and dried under vacuum at 60° C. to give anhydrous ciprofloxacin hydrochloride as a white solid (41.9 g, 114 mmol, 88% yield), giving the IR spectrum shown in FIG. 1 and the powder X-ray diffraction pattern of FIG. 3, bottom trace, peaks as listed on FIG. 4. The absence of any peaks attributable to hydration or solvent residue is to be noted.

### Example 2

Ciprofloxacin hydrochloride monohydrate (50.3 g, 130 mmol) was suspended in 2-butanol 350 mL and vigorously stirred for 2 hours. The suspension was then heated to reflux and 170 mL of liquid were distilled off. After cooling to room temperature, the solids were collected by filtration and dried under vacuum at 60° C. to give anhydrous ciprofloxacin hydrochloride as a white solid (42.3 g, 115 mmol, 88% yield). The product gave the same IR and X-ray spectra as that of Example 1.

### Example 3

Ciprofloxacin hydrochloride monohydrate was placed in an open vial, which in turn was placed inside a closed jar containing absolute ethanol. After 3 days, analysis of the solid by powder X-ray diffraction showed complete conversion into anhydrous ciprofloxacin hydrochloride, 100% yield. The product gave the same IR and X-ray spectra as that of Example 1.

What is claimed is:

1. An anhydrous and unsolvated crystalline polymorphic form of ciprofloxacin hydrochloride.

2. The polymorph of claim 1, characterized by XPRD peaks at 7.22, 9.26, 12.80, 15.71, 21.77 and 29.42 degrees 2-θ,

3. The polymorph of claim 1, wherein the polymorph exhibits an X-ray powder diffraction pattern substantially the same as that shown in FIG. 3, lower trace.

4. The polymorph of claim 1, wherein the polymorph exhibits an infrared spectrum substantially the same as that shown in FIG. 1.

5. The polymorph of claim 2, wherein the polymorph exhibits an infrared spectrum substantially the same as that shown in FIG. 1.

6. The polymorph of claim 3, wherein the polymorph exhibits an infrared spectrum substantially the same as that shown in FIG. 1.

7. A process of preparing an anhydrous, unsolvated crystalline polymorph of ciprofloxacin hydrochloride, comprising:
suspending ciprofloxacin hydrochloride monohydrate in a biocompatible solvent;
stirring the suspension;
collecting the resulting precipitate from the suspension by filtration;
drying the precipitate.

8. The process of claim 7, further comprising heating the suspension prior to filtration.

9. A process of preparing an anhydrous, unsolvated crystalline polymorph of ciprofloxacin hydrochloride, comprising:
placing a sample of ciprofloxacin hydrochloride monohydrate in a closed, saturated atmosphere of an organic biocompatible solvent, allowing the sample to equilibrate for a period of about 1-5 days so that the monohydrate converts to the desired polymorph, and recovering anhydrous, unsolvated crystalline ciprofloxacin hydrochloride.

10. The process of claim 7, wherein the biocompatible solvent is one which forms an azeotropic mixture with water.

11. The process of claim 8, wherein the biocompatible solvent is one which forms an azeotropic mixture with water.

12. The process of claim 9, wherein the biocompatible solvent is one which forms an azeotropic mixture with water.

13. The process of claim 7, wherein the solvent is ethanol, 1-butanol or 2-butanol.

14. The process of claim 8, wherein the solvent is ethanol, 1-butanol or 2-butanol.

15. The process of claim 9, wherein the solvent is ethanol, 1-butanol or 2-butanol.

16. A pharmaceutical composition comprising the polymorph in claim 1 and a pharmaceutically acceptable carrier.
